(19) United States
(12) Patent Application Publication
(10) Pub. No.: US 2012/0171163 A1
(43) Pub. Date: Jul. 5, 2012

(54) METHOD FOR INHIBITING A BACTERIAL INVASIVE MECHANISM USING A NUTRITIONAL COMPOSITION

(76) Inventors: Anja Wittke, Evansville, IN (US); Dattatreya Banavara, Evansville, IN (US); Robert J. McMahon, Evansville, IN (US); Cecilia Munoz, Evansville, IN (US)

(21) Appl. No.: 12/980,808

(22) Filed: Dec. 29, 2010

(51) Int. Cl. A61K 35/74; A61K 38/40; A61P 31/04

(52) U.S. Cl. 424/93.4; 424/93.45; 514/2.5

(57) ABSTRACT

A method for inhibiting a bacterial invasive mechanism involving administering to a human a nutritional composition including a lipid or fat; a protein source; about 5 to about 100 mg/100 kcal of a source of long chain polyunsaturated fatty acids which comprises docosahexanoic acid; about 0.1 to about 1 g/100 kcal of a prebiotic composition, wherein the prebiotic composition comprises at least 20% of an oligosaccharide; at least about 10 mg/100 kCal of lactoferrin selected from the group consisting of non-human lactoferrin, human lactoferrin produced by a genetically modified organism, and combinations thereof.

### TECHNICAL FIELD

This disclosure relates generally to the field of nutritional compositions, such as infant formulas, human milk fortifiers, children's dietary supplements, and the like, having active lactoferrin compositions therein. More particularly, the disclosure relates to a method for inhibiting the invasive mechanisms of harmful bacterial pathogens using a nutritional composition including one or more prebiotics and non-human lactoferrin and/or human lactoferrin produced by a genetically modified organism.

### BACKGROUND

There are currently a variety of dietary compositions for humans, especially young humans, to provide supplemental or primary nutrition at certain stages in life. Generally, commercial dietary compositions for infants seek to mimic to the extent possible the composition and associated functionality of human milk. Through a combination of proteins, some of which have physiological activity, and blended fat ingredients, dietary compositions are formulated such that they simulate human milk for use as a complete or partial substitute. Other ingredients often utilized in dietary compositions for infants may include a carbohydrate source such as lactose as well as other vitamins, minerals and elements believed to be present in human milk for the absorption by the infant.

Some of the proteins present within human milk provide for a defense against pathogens to prevent and inhibit infection while additionally promoting an immunological response within the infant. Proteins including haptocorrin and lactoferrin are understood to help infants defend against a variety of bacterial pathogens via bacteriostatic and bactericidal activity.

Lactoferrin is one of the primary proteins in human milk and is considered a glycoprotein having an average molecular weight of approximately 80 kilodaltons. It is an iron binding protein having the capacity to bind two molecules of iron in a reversible fashion and can facilitate the uptake of iron within the intestines for the human. Functionally, lactoferrin regulates iron absorption and as such can bind iron-based free radicals as well as donate iron for an immunological response.

An additional role of lactoferrin is its anti-microbial activity in guarding against intestinal infections in humans generally, but especially in infants. Lactoferrin has been known to be both bacteriostatic and bactericidal in inhibiting the growth of specific bacteria while also killing microbes prior to a successful invasion of intestinal cells.

In obtaining a commercially viable dietary composition, the addition of lactoferrin has generally been limited due to predicted losses of activity during processing. Generally, the temperature and pH requirements in processing infant formulas and other products such as human milk fortifiers and various children's products reduce specific functions of the lactoferrin, causing lactoferrin not to be included within a final formulation. In addition, lactoferrin is often considered only for its iron binding qualities; thus, lactoferrin may generally be excluded from a formulation where such properties are thought to be diminished by processing conditions.

Further, as known in the art, human breast milk is relatively low in iron, containing about 0.3 milligrams of iron per liter of breast milk. While this quantity is low, human infants have high absorption rate, absorbing about half of the iron from the breast milk. Alternatively, prior art formulas, with high levels of iron fortification, of from about 10 mg to about 12 milligrams per liter, human infants absorb less than about 5% of the total iron. With such increased levels of iron within the prior art formulas, virtually all of the iron binding sites are occupied as lactoferrin is a known iron transport protein.

Additional complications of the prior art formulas include the inability of providing a bacteriostatic effect. This is partially due to the use of lactoferrin with blocked or damaged binding sites as the bacteriostatic effect is at least partially related to the degree of binding to iron of the lactoferrin present within the formula.

Accordingly, it would be beneficial to provide a nutritional composition, such as an infant formula, human milk fortifier, children's dietary supplement, and the like, which contains lactoferrin having a bacteriostatic effect even after processing under conditions of high temperature and low pH. A combination of characteristics including the maintenance of the anti-invasion mechanisms of lactoferrin through either high or low pH or high temperature conditions, such as during pasteurization, provide for a dietary composition that may at least partially protect against harmful bacterial pathogens.

### BRIEF SUMMARY

Briefly, the present disclosure is directed, in an embodiment, to a method for inhibiting one or more of the invasive mechanisms of a bacterial pathogen using a nutritional composition comprising a lipid or fat, a protein source, a source of long chain polyunsaturated fatty acids which include docosahexanoic acid (DHA), a prebiotic composition, and non-human lactoferrin and/or human lactoferrin produced by a genetically modified organism, where the composition provides active anti-invasion mechanisms against strains of undesirable bacteria found in the human gut, even after processing which includes exposure to harsh environmental conditions.

The disclosure is also directed to a nutritional product comprising:

a. up to about 7 g/100 kcal of a fat or lipid, more preferably about 3 to about 7 g/100 kcal of a fat or lipid;

b. up to about 5 g/100 kcal of a protein source, more preferably about 1 to about 5 g/100 kcal of a protein source;

c. about 5 to about 100 mg/100 kcal of a source of long chain polyunsaturated fatty acids which include DHA, more preferably about 10 to about 50 mg/100 kcal of a source of long chain polyunsaturated fatty acids which include DHA;

d. about 1.0 to about 10.0 g/L of a prebiotic composition having at least 20% of an oligosaccharide; in one embodiment, the oligosaccharide comprises a combination of polydextrose and galacto-oligosaccharide. More preferably about 2.0 g/L to about 8.0 g/L of a prebiotic composition having at least 20% of an oligosaccharide which comprises a combination of polydextrose and galacto-oligosaccharide is present; and

e. at least about 10 mg/100 kCal of lactoferrin, more preferably about 70 mg to about 220 mg/100 kCal of lactoferrin, and most preferably about 90 mg to about 190 mg/100 kCal of lactoferrin.

Preferably, the lactoferrin is non-human lactoferrin and/or human lactoferrin produced by a genetically modified organism. In one particularly preferred embodiment, the lactoferrin used is such that an effective amount of a nutritional composition containing lactoferrin may be administered to inhibit at least one of the invasive mechanisms of at least one pathogen in the gastrointestinal tract of a human, even if, during processing, the nutritional composition has been exposed to pH and temperature fluctuations typical of certain processing conditions like pasteurization. Examples of such pathogens include Enterotoxigenic E. coli (ETEC), Enteropathogenic E. coli (EPEC), Haemophilus influenza, Shigatoxin producing E. coli (STEC), Enteroaggregative E. coli (EAEC), Salmonella ser. Typhimurium, Shigella flexneri, Rotavirus, Norovirus, Respiratory syncytial virus (RSV), Adenovirus, and combinations thereof.

Further embodiments include the method of promoting increased resistance to endotoxins comprising the administration of an effective amount of a dietary composition containing lactoferrin.

An object of the disclosure is a nutritional composition including lactoferrin having biological activity while maintaining stability in both acidic and high temperature environments.

Still another object of the disclosure is a method of protecting humans from the toxic effects of bacteria comprising administering an effective amount of lactoferrin having biological activity against human-borne undesirable bacteria, with bactericidal qualities.

These aspects and others that will become apparent to the skilled artisan upon review of the following description can be accomplished by providing a dietary composition comprising biologically active lactoferrin having stability in acidic and high temperature environments while retaining activity in the human gut. The dietary composition advantageously maintains biologically active lactoferrin despite processing conditions including both low pH and high temperature conditions. The lactoferrin includes anti-invasion mechanisms which may destruct the attachment factors and injection needle used by certain bacteria on human cells.

### DETAILED DESCRIPTION OF THE PREFERRED EMBODIMENTS

The present disclosure provides novel nutritional products that are easily digested, provide physiochemical benefits, and/or provide physiological benefits. In an embodiment of the present disclosure, a nutritional composition comprises a lipid or fat, a protein source, a source of long chain polyunsaturated fatty acids which include docosahexanoic acid (DHA), a prebiotic composition having at least 20% of an oligosaccharide, especially one which comprises galacto-oligosaccharide (GOS), and lactoferrin which provides active anti-invasion mechanisms against strains of undesirable bacteria found in the human gut. The lactoferrin included in the compositions is non-human lactoferrin and/or human lactoferrin produced by a genetically modified organism. In certain embodiments, the prebiotic comprises a combination of galacto-oligosaccharide and polydextrose. More particularly, the composition disclosed herein comprises:

a. up to about 7 g/100 kcal of a fat or lipid, more preferably about 3 to about 7 g/100 kcal of a fat or lipid;

b. up to about 5 g/100 kcal of a protein source, more preferably about 1 to about 5 g/100 kcal of a protein source;

c. about 5 to about 100 mg/100 kcal of a source of long chain polyunsaturated fatty acids which include DHA, more preferably about 10 to about 50 mg/100 kcal of a source of long chain polyunsaturated fatty acids which include DHA;

d. about 1.0 to about 10.0 g/L of a prebiotic composition having at least 20% of an oligosaccharide which comprises galacto-oligosaccharide, more preferably about 2.0 g/L to about 8.0 g/L of a prebiotic composition having at least 20% of galacto-oligosaccharide; and

e. at least about 10 mg/100 kCal of lactoferrin, more preferably about 70 mg to about 220 mg/100 kCal of lactoferrin, most preferably about 90 mg to about 190 mg/100 kCal of lactoferrin.

### DEFINITIONS

As used herein, the term “prebiotic” means a non-digestible food ingredient that beneficially affects the host by selectively stimulating the growth and/or activity of one or a limited number of bacteria in the colon that can improve the health of the host.

The term “probiotic” means a microorganism with low or no pathogenicity that exerts beneficial effects on the health of the host.

As used herein, the term “infant” is generally defined as a human from about birth to 12 months of age.

A “preterm infant” is an infant born after less than 37 weeks gestation.

A “full term infant” as used herein, means an infant born after at least 37 weeks gestation.

“Children” are defined as humans from the age of 12 months to about 12 years old.

An “anti-bacterially effective amount,” as used herein is generally defined as an amount of lactoferrin that provides at least one anti-invasion mechanism against strains of bacteria.

“Biologically active lactoferrin,” means lactoferrin which possesses at least one anti-invasion mechanism against pathogens.

The term “non-human lactoferrin”, as used herein, refers to lactoferrin having an amino acid sequence that is different from the amino acid sequence of human lactoferrin.

The term “organism”, as used herein, refers to any contiguous living system, such as animal, plant, fungus or micro-organism.

The term “simulating,” as used herein means having or taking the form or appearance of or having or producing a symptomatic resemblance to.

### Disclosure

In some embodiments, the nutritional product may be an infant formula. The term “infant formula” applies to a composition in liquid or powdered form that satisfies the nutrient requirements of an infant by being a substitute for human milk. In the United States, the content of an infant formula is dictated by the federal regulations set forth at 21 C.F.R. §§100, 106 and 107. These regulations define macronutrient, vitamin, mineral, and other ingredient levels in an effort to simulate the nutritional and other properties of human breast milk. In a separate embodiment, the nutritional product may be a human milk fortifier, meaning it is a composition which is added to human milk in order to enhance the nutritional value of human milk. As a human milk fortifier, the disclosed composition may be in powder or liquid form. In yet another embodiment, the disclosed nutritional product may be a children's nutritional composition.

The nutritional products of the disclosure may provide minimal, partial, or total nutritional support. The compositions may be nutritional supplements or meal replacements. In some embodiments, the compositions may be administered in conjunction with a food or nutritional composition. In this embodiment, the compositions can either be intermixed with the food or other nutritional compositions prior to ingestion by the subject or can be administered to the subject either before or after ingestion of a food or nutritional composition. The compositions may be administered to preterm infants receiving infant formula, breast milk, a human milk fortifier, or combinations thereof.

The compositions may, but need not, be nutritionally complete. The skilled artisan will recognize “nutritionally complete” to vary depending on a number of factors including, but not limited to, age, clinical condition, and dietary intake of the subject to whom the term is being applied. In general, “nutritionally complete” means that the nutritional composition of the present disclosure provides adequate amounts of all carbohydrates, lipids, essential fatty acids, proteins, essential amino acids, conditionally essential amino acids, vitamins, minerals, and energy required for normal growth. As applied to nutrients, the term “essential” refers to any nutrient which cannot be synthesized by the body in amounts sufficient for normal growth and to maintain health and which therefore must be supplied by the diet. The term “conditionally essential” as applied to nutrients means that the nutrient must be supplied by the diet under conditions when adequate amounts of the precursor compound is unavailable to the body for endogenous synthesis to occur.

The composition which is “nutritionally complete” for the preterm infant will, by definition, provide qualitatively and quantitatively adequate amounts of all carbohydrates, lipids, essential fatty acids, proteins, essential amino acids, conditionally essential amino acids, vitamins, minerals, and energy required for growth of the preterm infant. The composition which is “nutritionally complete” for the term infant will, by definition, provide qualitatively and quantitatively adequate amounts of all carbohydrates, lipids, essential fatty acids, proteins, essential amino acids, conditionally essential amino acids, vitamins, minerals, and energy required for growth of the term infant. The composition which is “nutritionally complete” for a child will, by definition, provide qualitatively and quantitatively adequate amounts of all carbohydrates, lipids, essential fatty acids, proteins, essential amino acids, conditionally essential amino acids, vitamins, minerals, and energy required for growth of a child.

The nutritional composition may be provided in any form known in the art, including a powder, a gel, a suspension, a paste, a solid, a liquid, a liquid concentrate, or a ready-to-use product. In one preferred embodiment, the nutritional composition is an infant formula, especially an infant formula adapted for use as sole source nutrition for an infant.

In the preferred embodiments, the nutritional product disclosed herein may be administered enterally. As used herein, “enteral” means through or within the gastrointestinal, or digestive, tract, and “enteral administration” includes oral feeding, intragastric feeding, transpyloric administration, or any other introduction into the digestive tract.

Suitable fat or lipid sources for practicing the present disclosure may be any known or used in the art, including but not limited to, animal sources, e.g., milk fat, butter, butter fat, egg yolk lipid; marine sources, such as fish oils, marine oils, single cell oils; vegetable and plant oils, such as corn oil, canola oil, sunflower oil, soybean oil, palmolein, coconut oil, high oleic sunflower oil, evening primrose oil, rapeseed oil, olive oil, flaxseed (linseed) oil, cottonseed oil, high oleic safflower oil, palm stearin, palm kernel oil, wheat germ oil; medium chain triglyceride oils and emulsions and esters of fatty acids; and any combinations thereof.

Bovine milk protein sources useful in practicing the present disclosure include, but are not limited to, milk protein powders, milk protein concentrates, milk protein isolates, nonfat milk solids, nonfat milk, nonfat dry milk, whey protein, whey protein isolates, whey protein concentrates, sweet whey, acid whey, casein, acid casein, caseinate (e.g. sodium caseinate, sodium calcium caseinate, calcium caseinate) and any combinations thereof.

In one embodiment, the proteins are provided as intact proteins. In other embodiments, the proteins are provided as a combination of both intact proteins and partially hydrolyzed proteins, with a degree of hydrolysis of between about 4% and 10%. In yet another embodiment, the protein source may be supplemented with glutamine-containing peptides.

In a particular embodiment of the disclosure, the whey:casein ratio of the protein source is similar to that found in human breast milk. In an embodiment, the protein source comprises from about 20% to about 80% whey protein. In another embodiment, the protein source may comprise from about 20% to about 80% caseins.

In one embodiment of the disclosure, the nutritional composition may contain one or more probiotics. Any probiotic known in the art may be acceptable in this embodiment provided it achieves the intended result. In a particular embodiment, the probiotic may be selected from Lactobacillus species, Lactobacillus rhamnosus GG, Bifidobacterium species, Bifidobacterium longum, Bifidobacterium brevis and Bifidobacterium animalis subsp. lactis BB-12.

If included in the composition, the amount of the probiotic may vary from about 10⁴ to about 10¹⁰ colony forming units (cfu) per kg body weight per day. In another embodiment, the amount of the probiotic may vary from about 10⁶ to about 10⁹ cfu per kg body weight per day. In yet another embodiment, the amount of the probiotic may be at least about 10⁶ cfu per kg body weight per day. Moreover, the disclosed composition may also include probiotic-conditioned media components.

In an embodiment, the probiotic(s) may be viable or non-viable. As used herein, the term “viable”, refers to live microorganisms. The term “non-viable” or “non-viable probiotic” means non-living probiotic microorganisms, their cellular components and metabolites thereof. Such non-viable probiotics may have been heat-killed or otherwise inactivated but retain the ability to favorably influence the health of the host. The probiotics useful in the present disclosure may be naturally-occurring, synthetic or developed through the genetic manipulation of organisms, whether such new source is now known or later developed.

The nutritional composition contains one or more prebiotics. Such prebiotics may be naturally-occurring, synthetic, or developed through the genetic manipulation of organisms and/or plants, whether such new source is now known or developed later. In certain embodiments, the prebiotic included in the compositions of the present disclosure include those taught by U.S. Pat. No. 7,572,474, the disclosure of which is incorporated herein by reference.

Prebiotics useful in the present disclosure may include oligosaccharides, polysaccharides, and other prebiotics that contain fructose, xylose, soya, galactose, glucose and mannose. More specifically, prebiotics useful in the present disclosure may include lactulose, lactosucrose, raffinose, gluco-oligosaccharide, inulin, polydextrose, polydextrose powder, galacto-oligosaccharide, fructo-oligosaccharide, isomalto-oligosaccharide, soybean oligosaccharides, lactosucrose, xylo-oligosacchairde, chito-oligosaccharide, manno-oligosaccharide, aribino-oligosaccharide, siallyl-oligosaccharide, fuco-oligosaccharide, and gentio-oligosaccharides.

In an embodiment, the total amount of prebiotics present in the nutritional composition may be from about 1.0 g/L to about 10.0 g/L of the composition. As noted, the total amount of prebiotics present in the nutritional composition may be from about 2.0 g/L and about 8.0 g/L of the composition. At least 20% of the prebiotics should comprise galacto-oligosaccharide (GOS).

In addition to galacto-oligosaccharide, the prebiotic composition can also comprise polydextrode (PDX). If polydextrose is used as a prebiotic, the amount of polydextrose in the nutritional composition may, in an embodiment, be within the range of from about 1.0 g/L to about 4.0 g/L.

The amount of galacto-oligosaccharide in the nutritional composition may, in an embodiment, be from about 0.2 g/100 Kcal to about 1.0 g/100 Kcal. In another embodiment, the amount of galacto-oligosaccharide in the nutritional composition may be from about 0.1 g/100 Kcal to about 0.5 g/100 Kcal. If polydextrose is used as a prebiotic, the amount of polydextrose in the nutritional composition may, in an embodiment, be within the range of from about 0.1 g/100 Kcal to about 0.5 g/100 Kcal.

The nutritional formulation of the disclosure contains a source of long chain polyunsaturated fatty acids (LCPUFAs) which comprise docosahexanoic acid (DHA). Other suitable LCPUFAs include, but are not limited to, α-linoleic acid, γ-linoleic acid, linoleic acid, linolenic acid, eicosapentanoic acid (EPA) and arachidonic acid (ARA).

In one embodiment, the nutritional composition is supplemented with both DHA and ARA. In this embodiment, the weight ratio of ARA:DHA may be from about 1:3 to about 9:1. In one embodiment of the present disclosure, this ratio is from about 1:2 to about 4:1.

The amount of long chain polyunsaturated fatty acids in the nutritional composition may vary from about 5 mg/100 kcal to about 100 mg/100 kcal, more preferably from about 10 mg/100 kcal to about 50 mg/100 kcal.

The nutritional composition may be supplemented with oils containing DHA and ARA using standard techniques known in the art. For example, DHA and ARA may be added to the formula by replacing an equivalent amount of an oil, such as high oleic sunflower oil, normally present in the formula. As another example, the oils containing DHA and ARA may be added to the formula by replacing an equivalent amount of the rest of the overall fat blend normally present in the formula without DHA and ARA.

If utilized, the source of DHA and ARA may be any source known in the art such as marine oil, fish oil, single cell oil, egg yolk lipid, and brain lipid. In some embodiments, the DHA and ARA are sourced from the single cell Martek oil, DHASCO® and ARASCO® respectively, or variations thereof. The DHA and ARA can be in natural form, provided that the remainder of the LCPUFA source does not result in any substantial deleterious effect on the infant. Alternatively, the DHA and ARA can be used in refined form.

In an embodiment of the present disclosure, sources of DHA and ARA are single cell oils as taught in U.S. Pat. Nos. 5,374,567; 5,550,156; and 5,397,591, the disclosures of which are incorporated herein in their entirety by reference. However, the present disclosure is not limited to only such oils.

Lactoferrins are single chain polypeptides of about 80 kD containing 1-4 glycans, depending on the species. The 3-D structures of lactoferrin of different species are very similar, but not identical. Each lactoferrin comprises two homologous lobes, called the N- and C-lobes, referring to the N-terminal and C-terminal part of the molecule, respectively. Each lobe further consists of two sub-lobes or domains, which form a cleft where the ferric ion (Fe³⁺) is tightly bound in synergistic cooperation with a (bi)carbonate anion. These domains are called N1, N2, C1 and C2, respectively. The N-terminus of lactoferrin has strong cationic peptide regions that are responsible for a number of important binding characteristics. Lactoferrin has a very high isoelectric point (˜pI 9) and its cationic nature plays a major role in its ability to defend against bacterial, viral, and fungal pathogens. There are several clusters of cationic amino acids residues within the N-terminal region of lactoferrin mediating the biological activities of lactoferrin against a wide range of microorganisms. For instance, the N-terminal residues 1-47 of human lactoferrin (1-48 of bovine lactoferrin) are critical to the iron-independent biological activities of lactoferrin. In human lactoferrin, residues 2 to 5 (RRRR) and 28 to 31 (RKVR) are arginine-rich cationic domains in the N-terminus especially critical to the antimicrobial activities of lactoferrin. A similar region in the N-terminus is found in bovine lactoferrin (residues 17 to 42; FKCRRWQWRMKKLGAPSITCVRRAFA).

As described in “Perspectives on Interactions Between Lactoferrin and Bacteria” which appeared in the publication BIOCHEMISTRY AND CELL BIOLOGY, pp 275-281 (2006), lactoferrins from different host species may vary in their amino acid sequences though commonly possess a relatively high isoelectric point with positively charged amino acids at the end terminal region of the internal lobe. Suitable lactoferrins for use in the present disclosure include those having at least 48% homology with the amino acid sequence AVGEQELRKCNQWSGL at the HLf (349-364) fragment. In some embodiments, the lactoferrin has at least 65% homology with the amino acid sequence AVGEQELRKCNQWSGL at the HLf (349-364) fragment, and, in embodiments, at least 75% homology. For example, non-human lactoferrins for use in the present disclosure include, without limitation, bovine lactoferrin, porcine lactoferrin, equine lactoferrin, buffalo lactoferrin, goat lactoferrin, murine lactoferrin and camel lactoferrin.

Surprisingly, the forms of lactoferrin included herein maintain relevant activity even if exposed to a low pH (i.e., below 7, and even as low as about 4.6 or lower) and/or high temperatures (i.e., above about 65° C., and as high as about 120° C., conditions which would be expected to destroy or severely limit the stability or activity of human lactoferrin or recombinant human lactoferrin. These low pH and/or high temperature conditions can be expected during certain processing regimen for nutritional compositions of the types described herein, such as pasteurization. For instance, while bovine lactoferrin has an the amino acid composition which has only about a 70% sequence homology to that of human lactoferrin, and is stable and remains active under conditions under which human or recombinant human lactoferrin become unstable or inactive, bovine lactoferrin has bactericidal activity against undesirable bacterial pathogens found in the human gut.

In U.S. Pat. No. 4,791,193, incorporated by reference herein in its entirety, Okonogi et al. discloses a process for producing bovine lactoferrin in high purity. Generally, the process as disclosed includes three steps. Raw milk material is first contacted with a weakly acidic cationic exchanger to absorb lactoferrin followed by the second step where washing takes place to remove nonabsorbed substances. A desorbing step follows where lactoferrin is removed to produce purified bovine lactoferrin. Other methods may include steps as described in U.S. Pat. Nos. 7,368,141, 5,849,885, 5,919,913 and 5,861,491, the disclosures of which are all incorporated by reference in their entirety.

A benefit of lactoferrin as used in embodiments of the present disclosure is its anti-invasion mechanism in the human gut. Specifically, lactoferrin may destroy the injection needle used by certain bacteria to invade and cause pathogenesis. One such example of a bacterium known to cause pathogenesis is Escherichia coli which may cause diarrhea in infants, children and adults and is realized as an agent for pediatric diarrhea. The E. coli produces and translocates bacterial protein through a needle complex via a type III secretory system.

The secretory system of many gram-negative pathogenic bacteria is a type III secretion including the following bacteria: Shigella, Salmonella, Pseudomonas, and Escherichia coli. The type III secretory system functions through use of a needle for the transport of virulent proteins from the bacterial cytoplasm through the needle directly into the host cell's cytoplasm. The use of the needle provides for a passage through the multiple membranes including the double membranes of the gram-negative bacterium and the eukaryotic membrane of the human cell. Specifically, in strains of E. coli the needle complex is comprised of E. coli secretion component F (EscF) with E. coli secreted protein A (EspA) attaching to the tip of the needle, forming a generally hollow structure for the passage of components from the bacteria to the host human cell. At this point, bacterial proteins such as EspB may be introduced into the host cell through this tube. While the physiology of EspB may not be fully understood in the article “The Enteropathogenic E. coli effector EspB facilitates microvillus effacing and Antiphagocytosis by Inhibiting Myosin Function” in CELL HOSTS AND MICROBE, pp 383-392 (2007), EspB is described as binding to myosins which ultimately suppresses phagocytosis as a human immune response. Generally, myosin proteins interact with actin filaments to participate in cellular processes such as phagocytosis in eliminating potential bacterial pathogens. Harmful symptoms occur where EspB emitted by the E. coli inhibits the interaction between various myosin proteins and actin filaments in suppressing phagocytosis, leading to diarrhea or other gastric distress in infants, children and adults.

One of the anti-invasion mechanisms of lactoferrin is in inhibiting the translocation of EspB into human cells. Specifically, one mechanism may include the inhibition of the formation of the necessary secretory structures for translocating EspB from the bacteria. Lactoferrin is capable of degrading EspA, the protein responsible for the tube like structure for translocating Esp B into the host cell. As EspA may be degraded by lactoferrin, the portal through the human cell membrane would not be created thus alleviating pathogenesis created from Esp B entering into the human cell's cytoplasm. Furthermore, lactoferrin may also possess proteolytic activity resulting in the degrading EspB. Ultimately, lactoferrin effectively disrupts the needle complex associated with the pathogen's secretion system while simultaneously degrading proteins responsible for symptoms including gastrointestinal distress and diarrhea.

In one embodiment, lactoferrin is present in the nutritional composition in an amount of at least about 10 mg/100 kCal, especially when the nutritional composition is intended for use by children. In certain embodiments, the upper limit of lactoferrin is about 240 mg/100 kCal. In another embodiment, where the nutritional composition is an infant formula, lactoferrin is present in the nutritional composition in an amount of from about 70 mg to about 220 mg/100 kCal; in yet another embodiment, lactoferrin is present in an amount of about 90 mg to about 190 mg/100 kCal. Nutritional compositions for infants can include lactoferrin in the quantities of from about 0.5 mg to about 1.5 mg per milliliter of formula. In nutritional compositions replacing human milk, lactoferrin may be present in quantities of from about 0.6 mg to about 1.3 mg per milliliter of formula.

### EXAMPLES

The following examples are provided to illustrate embodiments of the nutritional composition of the present disclosure but should not be interpreted as any limitation thereon. Other embodiments within the scope of the claims herein will be apparent to one skilled in the art from the consideration of the specification or practice of the nutritional composition or methods disclosed herein. It is intended that the specification, together with the example, be considered to be exemplary only, with the scope and spirit of the disclosure being indicated by the claims which follow the examples.

### Example 1

This example illustrates an embodiment of a nutritional product according to the present disclosure.

| Description | kg per 100 kg |
|---|---|
| carbohydrate, total | 38.9 |
| protein, total | 28.8 |
| fat, total | 25.6 |
| prebiotics | 4.5 |
| soy lecithin | 0.8 |
| lactoferrin | 0.3 |
| calcium carbonate | 0.5 |
| potassium citrate | 0.2 |
| ferrous sulfate | 0.05 |
| potassium chloride | 0.048 |
| magnesium oxide | 0.023 |
| sodium chloride | 0.025 |
| zinc sulfate | 0.015 |
| cupric sulfate | 0.002 |
| manganese sulfate | 0.0003 |
| sodium selenite | 0.00003 |
| choline chloride | 0.144 |
| ascorbic acid | 0.093 |
| Niacinamide | 0.006 |
| calcium pantothenate | 0.003 |
| vitamin A palmitate | 0.007 |
| vitamin B12 | 0.002 |
| vitamin D3 | 0.000001 |
| Riboflavin | 0.0008 |
| thiamin | 0.0006 |
| vitamin B6 | 0.0004 |
| folic acid | 0.0001 |
| vitamin K1 | 0.006 |
| biotin | 0.00002 |
| inositol | 0.03 |
| vitamin E acetate | 0.01 |
| taurine | 0.05 |
| L-carnitine | 0.001 |

### Example 2

This example illustrates another embodiment of a nutritional product according to the present disclosure.

| Description | kg per 100 kg |
|---|---|
| carbohydrate, total | 24.7 |
| protein, total | 31.9 |
| fat, total | 39.3 |
| prebiotics | 3.6 |
| lactoferrin | 0.1 |
| calcium carbonate | 0.15 |
| ferrous sulfate | 0.03 |
| zinc sulfate | 0.01 |
| copper sulfate | 0.00025 |
| manganese sulfate | 0.0002 |
| sodium selenite | 0.00001 |
| choline bitartrate | 0.05 |
| ascorbic acid | 0.004 |
| sodium ascorbate | 0.04 |
| niacinamide | 0.007 |
| calcium pantothenate | 0.0005 |
| vitamin A palmitate | 0.0005 |
| vitamin D3 | 0.0002 |
| riboflavin | 0.0001 |
| thiamin | 0.00005 |
| vitamin B6 | 0.00005 |
| folic acid | 0.000067 |
| vitamin K1 | 0.00002 |
| vitamin E acetate | 0.008 |
| taurine | 0.02 |
| fish oil | 0.2 |
| B-glucan | 0.03 |

### Example 3

This example illustrates one embodiment of ingredients that can be used to prepare the nutritional product according to the present disclosure.

| | | |
|---|---|---|
| water | 872 | ml |
| lactose | 65.6 | mg |
| vegetable oil blend | 353.0 | mg |
| nonfat milk evaporated | 34.0 | mg |
| whey protein concentrate | 8.5 | mg |
| galacto-oligosaccharide | 4.7 | mg |
| casein | 3.5 | mg |
| polydextrose | 2.4 | mg |
| lactoferrin solution (10%) | 1.0 | mg |
| single cell DHA and ARA oil blend | 0.94 | mg |
| mono- and di-glycerides | 0.7 | mg |
| calcium carbonate | 0.44 | mg |
| calcium phosphate | 0.4 | mg |
| potassium citrate | 0.4 | mg |
| potassium chloride | 0.4 | mg |
| soy lecithin | 0.4 | mg |
| sodium chloride | 0.3 | mg |
| potassium phosphate | 0.3 | mg |
| choline chloride | 0.2 | mg |
| magnesium oxide | 0.08 | mg |
| calcium hydroxide | 0.08 | mg |
| ferrous suflate | 0.07 | mg |

### Example 4

This example illustrates another embodiment of ingredients that can be used to prepare the nutritional product according to the present disclosure.

| | | |
|---|---|---|
| water | 686 | ml |
| reduced minerals whey | 215 | mg |
| nonfat milk evaporated | 67 | mg |
| vegetable oil blend | 33 | mg |
| lactose | 17 | mg |
| galacto-oligosaccharide | 4.7 | mg |
| polydextrose | 2.4 | mg |
| lactoferrin solution (10%) | 1.0 | mg |
| single cell DHA and ARA oil blend | 0.9 | mg |
| mono- and di-glycerides | 0.7 | mg |
| calcium carbonate | 0.44 | mg |
| calcium phosphate | 0.4 | mg |
| potassium citrate | 0.4 | mg |
| potassium chloride | 0.4 | mg |
| soy lecithin | 0.4 | mg |
| potassium phosphate | 0.3 | mg |
| carrageenan | 0.3 | mg |
| sodium citrate | 0.2 | mg |
| choline chloride | 0.2 | mg |
| magnesium oxide | 0.08 | mg |
| calcium chloride | 0.08 | mg |
| ferrous suflate | 0.07 | mg |

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although preferred embodiments of the disclosure have been described using specific terms, devices, and methods, such description is for illustrative purposes only. The words used are words of description rather than of limitation. It is to be understood that changes and variations may be made by those of ordinary skill in the art without departing from the spirit or the scope of the present disclosure, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the preferred versions contained therein.

What is claimed is:

1. A method for inhibiting at least one invasion mechanism of a bacterial pathogen comprising administering to a human a nutritional composition, comprising
a. a lipid or fat;
b. a protein source;
c. about 5 to about 100 mg/100 kcal of a source of long chain polyunsaturated fatty acids which comprises docosahexanoic acid;
d. about 0.1 to about 1 g/100 kcal of a prebiotic composition, wherein the prebiotic composition comprises at least 20% of an oligosaccharide;
e. at least about 10 mg/100 kCal of lactoferrin selected from the group consisting of non-human lactoferrin, human lactoferrin produced by a genetically modified organism, and combinations thereof.

2. The method of claim 1, wherein the source of long chain polyunsaturated fatty acids further comprises arachidonic acid.

3. The method of claim 1, wherein the lipid or fat is present at a level of up to about 7 g/100 kcal.

4. The method of claim 1, wherein the protein source is present at a level of up to about 5 g/100 kcal.

5. The method of claim 1, wherein the oligosaccharide comprises galacto-oligosaccharide.

6. The method of claim 5, wherein the oligosaccharide further comprises polydextrose.

7. The method of claim 1, which further comprises at least one probiotic.

8. The method of claim 7, wherein the probiotic is selected from the group consisting of Bifidobacteria spp., Lactobacillus spp and combinations thereof.

9. The method of claim 1, wherein the lactoferrin has at least 48% homology with the amino acid sequence AVGEQELRKCNQWSGL at the HLf (349-364) fragment.

10. The method of claim 9, wherein the lactoferrin is bovine lactoferrin.

11. The method of claim 9, wherein the bovine lactoferrin is stable and remains active under conditions under which human or recombinant human lactoferrin become unstable or inactive.

12. The method of claim 1, wherein the lactoferrin is present at a level of about 70 mg to about 220 mg/100 kCal.

13. A nutritional composition having anti-microbial properties in the human gut, comprising
a. a lipid or fat;
b. a protein source;
c. about 5 to about 100 mg/100 kcal of a source of long chain polyunsaturated fatty acids which comprises docosahexanoic acid;
d. about 0.1 to about 1 g/100 kcal of a prebiotic composition, wherein the prebiotic composition comprises at least 20% of an oligosaccharide;
e. at least about 10 mg/100 kCal of lactoferrin selected from the group consisting of non-human lactoferrin, human lactoferrin produced by a genetically modified organism, and combinations thereof.

14. The nutritional composition of claim 13, wherein the lipid or fat is present at a level of up to about 7 g/100 kcal.

15. The nutritional composition of claim 13, wherein the protein source is present at a level of up to about 5 g/100 kcal.

16. The nutritional composition of claim 13, wherein the oligosaccharide comprises galacto-oligosaccharide.

17. The nutritional composition of claim 16, wherein the oligosaccharide further comprises polydextrose.

18. The nutritional composition of claim 13, which further comprises at least one probiotic.

19. The nutritional composition of claim 13, wherein the lactoferrin has at least 48% homology with the amino acid sequence AVGEQELRKCNQWSGL at the HLf (349-364) fragment.

20. The nutritional composition of claim 13, wherein the lactoferrin is present at a level of about 70 mg to about 220 mg/100 kCal.
